(19) United States
(12) Patent Application Publication
(10) Pub. No.: US 2010/0022798 A1
(43) Pub. Date: Jan. 28, 2010

(54) Ibuprofen amine salts and synthesis thereof

(75) Inventors: Tu Lee, Flushing, NY (US); Yeh-Wen Wang, Taoyuan County (TW)

Correspondence Address: HUDAK, SHUNK & FARINE, CO., L.P.A., 2020 FRONT STREET, SUITE 307, CUYAHOGA FALLS, OH, 44221, US

(73) Assignee: NATIONAL CENTRAL UNIVERSITY, Jhongli City (TW)

(21) Appl. No.: 12/380,207

(22) Filed: Feb. 25, 2009

(30) Foreign Application Priority Data
Jul. 25, 2008 (TW) 097128214

(51) Int. Cl. C07B 55/00; C07C 53/134

(52) U.S. Cl. 562/401; 562/496

(57) ABSTRACT

The present invention discloses an ibuprofen amine salt and the synthesis thereof, and more particularly an ibuprofen amine salt yielded by the neutralization reaction of a racemic mixture of ibuprofen with tris(hydroxymethyl)aminomethane in a solution system of water and an organic solvent. Compared with racemic mixtures of ibuprofen, the ibuprofen amine salt has higher solubility and a higher melting point, thereby having better bioavailability and properties than racemic mixtures of ibuprofen during pharmaceutical processing. Further, the ibuprofen amine salts of the present invention are stable between pH 4 and 9 so that they can be more widely applied to medicines.

### BACKGROUND OF THE INVENTION

(a) Field of the Invention

The present invention relates to an ibuprofen amine salt and the synthesis thereof, and more particularly to an ibuprofen amine salt yielded by the neutralization reaction of a racemic mixture of ibuprofen with tris(hydroxymethyl)aminomethane in a solution system of water and an organic solvent.

(b) Description of the Prior Art

Ibuprofen, i.e. 2-(4-isobutylphenyl)-propionic acid is a known medicine with analgesic, antiphlogistic and antipyretic properties, that in particular is employed for the treatment of inflammatory diseases and against pain, such as rheumatic diseases, headaches, migraines, toothaches, back aches, muscle pain, postoperative pain and the like. The therapeutically effective form is the S(+)-ibuprofen, whereas the R(−)-enantiomer is practically ineffective, but converts in the body partly into the effective S(+)-form. However, ibuprofen is still employed mostly in racemic form.

The bioavailability of the ibuprofen medicaments including those in racemic form is limited by the poor solubility of the medicament in aqueous body fluid after administration. Hence, for rapidly reaching the therapeutically effective concentration of the medicament, the rate-limiting step becomes a critical step.

Therefore, a method for solving the poor solubility of the medicament after administration includes the use of a large amount of aqueous diluents, solubilizers, detergents, non-aqueous solvents or solutions at non-physiological pH values in the prior technology. However, such formulations will increase the systematic toxicity of pharmaceutical compositions and damage the body tissues administered.

With the addition of a large amount of accessory ingredients, ibuprofen is usually converted into a sufficiently stable solid dosage form such as a tablet. But in the presence of a large amount of accessory ingredients, the concentrates containing the ibuprofen active ingredient are limited to 30%-50%, thus resulting in hard-to-swallow larger tablets as well as higher manufacturing costs. Furthermore, an ibuprofen amine in racemic form has a lower melting point so that a smaller solid dosage form cannot be obtained during tablet compression.

For this reason, how to increase the solubility of ibuprofen in racemic form to reduce the addition of accessory ingredients and elevate the melting point of ibuprofen in racemic form thereby obtaining a smaller solid dosage form is an issue eager to be solved.

In order to resolve all the problems of the prior art, the inventors propose an ibuprofen amine salt and the synthesis thereof based on their research and development for many years and plenty of practical experiences, thereby improving the above shortcomings.

### SUMMARY OF THE INVENTION

In view of the above-mentioned circumstances, an objective of the present invention is to provide an ibuprofen amine salt and the synthesis thereof, thereby solving the problems of low solubility and low melting point as described above.

According to the objective of the present invention, an ibuprofen amine salt is characterized in that the ibuprofen amine salt is yielded by the neutralization reaction of a racemic mixture of ibuprofen with tris(hydroxymethyl)aminomethane in a solution system of water and an organic solvent.

Furthermore, the present invention provides a method of synthesizing an ibuprofen amine salt, comprising the steps of: providing a racemic mixture of ibuprofen and an aqueous solution of tris(hydroxymethyl)aminomethane, dissolving a first number of moles of said racemic mixture of ibuprofen into an organic solvent to obtain a mixed solution, adding an aqueous solution of a second number of moles of tris(hydroxymethyl)aminomethane to the mixed solution, then performing a stirring process and subsequently performing a separation process to obtain a precipitate, then performing a washing process on the precipitate with an aqueous solution containing an organic solvent, and finally performing a drying process to obtain the ibuprofen amine salt.

Moreover, the present invention provides a method of synthesizing an ibuprofen amine salt, comprising the steps of: providing a racemic mixture of ibuprofen, tris(hydroxymethyl)aminomethane and an organic solvent, then adding a first number of moles of the racemic mixture of ibuprofen and a first number of moles of the tris(hydroxymethyl)aminomethane to water and remaining in a constant temperature environment of a first temperature, after adding a second number of moles of the organic solvent, performing a stirring process until the color of the solution is near clear, then placing the solution in a constant temperature environment of a second temperature until the crystal is crystallized from the solution, then performing a separation process to obtain the crystal, and finally performing a drying process to obtain the ibuprofen amine salt.

The technical features and effects of the present invention may be further understood and appreciated from the following detailed description of the preferred embodiments.

### BRIEF DESCRIPTION OF THE DRAWINGS

Ibuprofen amine salts and the syntheses thereof according to preferred embodiments of the present invention are described below with reference to the related drawings where the same elements are designated with the same reference numerals for the convenience of understanding.

FIG. 1 is a flow chart showing the steps of a method of synthesizing an ibuprofen amine salt according to the present invention;

FIG. 2 is a flow chart showing the steps of a method of synthesizing an ibuprofen amine salt according to the present invention;

FIG. 3 is a temperature scan of an ibuprofen amine salt according to the present invention obtained by differential scanning calorimetry;

FIG. 4 is an analytical chart of an ibuprofen amine salt according to the present invention obtained by infrared spectroscopy; and

FIG. 5 is an X-ray powder diffractogram of an ibuprofen amine salt according to the present invention.

### DETAILED DESCRIPTION OF THE PREFERRED EMBODIMENTS

Generally speaking, the organic molecules of medicines have a poor solubility in water. In the pharmaceutical industry, medicines are often transformed into the solid forms of their “salts”, thereby improving their physical and chemical properties, such as solubility, stability, bioavailability, workability, taste, and the like.

Therefore, the present invention provides an ibuprofen amine salt, characterized in that the ibuprofen amine salt is yielded by the neutralization reaction of a racemic mixture of ibuprofen with tris(hydroxymethyl)aminomethane in a solution system of water and an organic solvent. Wherein the organic solvent may comprise acetone, methanol, dimethyl sulfoxide, ethanol, N,N-dimethylformamide, acetonitrile, isopropyl alcohol, 1,4-dioxane or tetrahydrofuran. The crystal of the ibuprofen amine salt is white and the aspect ratio of the crystal thereof ranges from 0.9 to 1.1. The solubility of the ibuprofen amine salt in water ranges from 5.5 mg/ml to 6.5 mg/ml. The melting point ranges from 158□ to 162□, and the density ranges from 1.1 g/cm³ to 1.3 g/cm³.

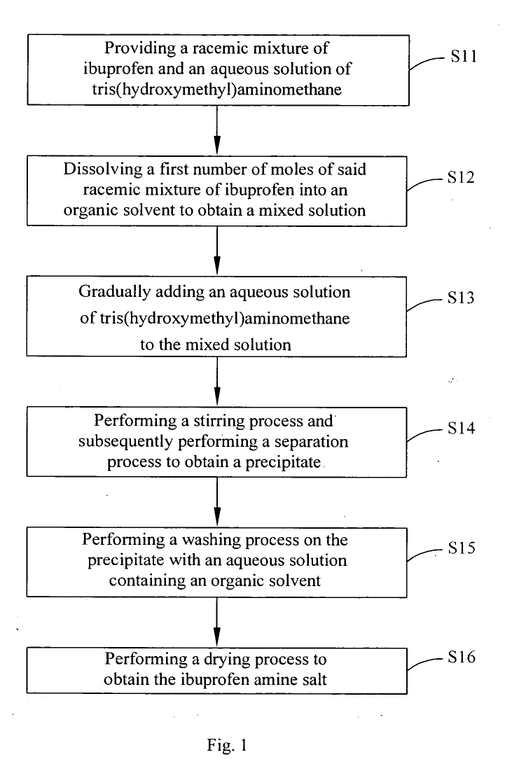

Referring to FIG. 1, there is a flow chart showing the steps of a method of synthesizing an ibuprofen amine salt according to the present invention. In the drawing, this method comprises the following steps:

Step S11: providing a racemic mixture of ibuprofen and an aqueous solution of tris(hydroxy-methyl)aminomethane.

Step S12: dissolving a first number of moles of the racemic mixture of ibuprofen into an organic solvent to obtain a first solution.

Wherein in order to enable the racemic mixture of ibuprofen to completely form a precipitate, an excess of the aqueous solution of tris(hydroxymethyl)-aminomethane must be added so that the second number of moles must be greater than the first number of moles.

Step S13: gradually adding an aqueous solution of tris(hydroxymethyl)aminomethane to the mixed solution.

Step S14: performing a stirring process and subsequently performing a separation process to obtain a precipitate.

In actual operation, the described separation process can include filtration, centrifugation, or volatilization, and the obtained precipitate is white.

Step S15: performing a washing process on the precipitate with an aqueous solution containing an organic solvent.

Wherein the aqueous solution containing an organic solvent may comprise an aqueous solution of acetone, methanol, dimethyl sulfoxide, ethanol, N,N-dimethylformamide, acetonitrile, isopropyl alcohol, 1,4-dioxane or tetrahydrofuran.

Step S16: performing a drying process to obtain the ibuprofen amine salt.

The obtained ibuprofen amine salt is analyzed. As a result, the aspect ratio of the crystal thereof ranges from 0.9 to 1.1, the solubility in water ranges from 5.5 mg/ml to 6.5 mg/ml, the melting point ranges from 158□ to 162□, and the density ranges from 1.1 g/cm³ to 1.3 g/cm³.

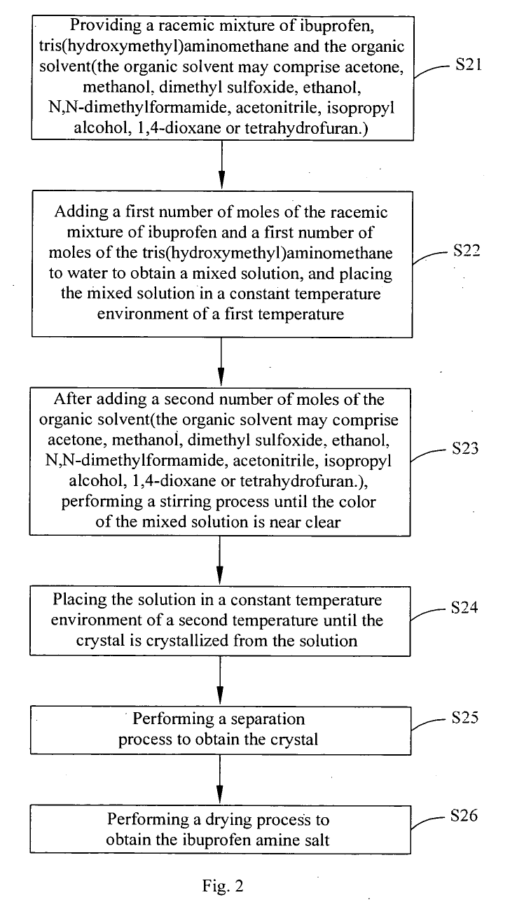

Referring to FIG. 2, there is depicted a flow chart showing the steps of a method of synthesizing an ibuprofen amine salt according to the present invention. In the drawing, this method comprises the following steps:

Step S21: providing a racemic mixture of ibuprofen, tris(hydroxymethyl)aminomethane and an organic solvent.

Wherein the organic solvent may comprise acetone, methanol, dimethyl sulfoxide, ethanol, N,N-dimethylformamide, acetonitrile, isopropyl alcohol, 1,4-dioxane or tetrahydrofuran.

Step S22: adding a first number of moles of the racemic mixture of ibuprofen and a first number of moles of the tris(hydroxymethyl)aminomethane to water to obtain a mixed solution, and placing the mixed solution in a constant temperature environment of a first temperature.

Wherein in order to enable the racemic mixture of ibuprofen to completely form a precipitate, an excess of tris(hydroxymethyl)aminomethane must be added so that the second number of moles must be greater than the first number of moles. The first temperature ranges from 45° C. to 55° C., and the reaction energy provided by heating the mixed solution permits the reaction velocity to be increased.

Step S23: after adding a second number of moles of the organic solvent, performing a stirring process until the color of the mixed solution is near clear.

Step S24: placing the solution in a constant temperature environment of a second temperature until the crystal is crystallized from the solution.

Wherein the described second temperature ranges from 15° C. to 35° C. The solubility of the solution can be reduced to facilitate the crystal is crystallized out by lowering the temperature of the solution.

Step S25: performing a separation process to obtain the crystal.

In actual operation, the described separation process can include filtration, centrifugation, or volatilization, and the obtained crystal is white.

Step S26: performing a drying process to obtain the ibuprofen amine salt.

The obtained ibuprofen amine salt is analyzed. As a result, the aspect ratio of the crystal thereof ranges from 0.9 to 1.1, the solubility in water ranges from 5.5 mg/ml to 6.5 mg/ml, the melting point ranges from 158° C. to 162° C., and the density ranges from 1.1 g/cm³ to 1.3 g/cm³.

The tests such as differential scanning calorimetry analysis, Fourier transform infrared spectroscopic analysis, X-ray powder diffraction analysis, and single crystal diffraction analysis etc. are carried out on the ibuprofen amine salt of the present invention as below.

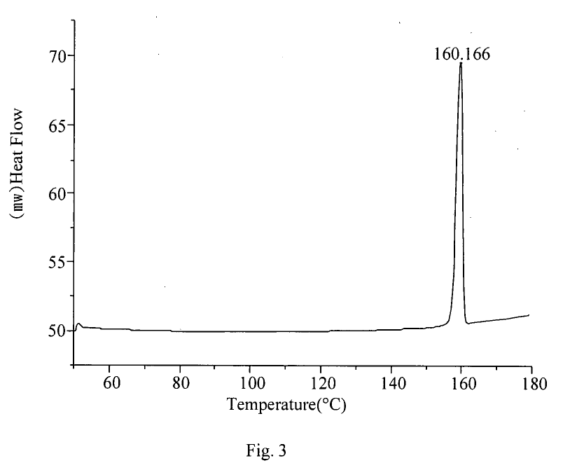

Referring to FIG. 3, there is depicted a temperature scan of an ibuprofen amine salt according to the present invention obtained by differential scanning calorimetry. A sample is placed under a specific atmosphere and the ambient temperature is changed or maintained at a constant value to observe the variation of energy of the sample when a differential scanning calorimetry analysis is performed. When physical phenomena or chemical changes such as melting, evaporation, crystallization, and phase transition, and the like occur in the sample, endothermic or exothermic bands will be present in its spectra, thereby predicting the thermal properties of the sample. In FIG. 3, an endothermic peak temperature ranges from 158° C. to 161° C. Therefore, it can be presumed that the melting point of the ibuprofen amine salt according to the present invention is between 158° C. and 161° C.

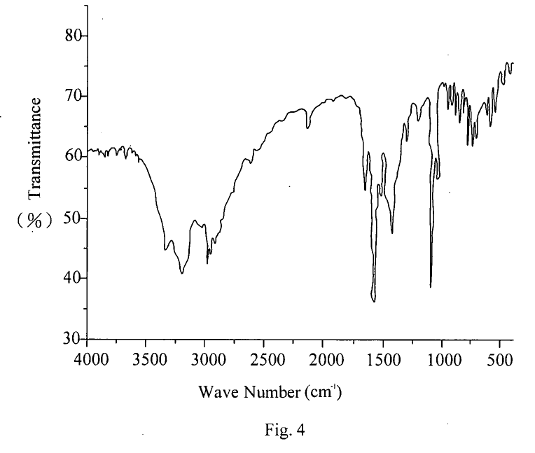

Referring to FIG. 4, there is depicted an analytical chart of an ibuprofen amine salt according to the present invention obtained by infrared spectroscopy. If the atoms in organic compound molecules vibrate and their vibration frequency is the same as the frequency of the infrared spectrum, thereby generating a dipole moment, the atoms will absorb the infrared rays and produce an infrared spectrum. Therefore, according to the vibration frequency of the generated infrared spectrum, it can be observed that the wave number ranges from 4000 cm⁻¹ to 400 cm⁻¹ from the infrared absorption spectrum of the ibuprofen amine salt according to the present invention. From this drawing, it can be seen that the compound contains a CH₂—OH structure at a wave number of 1000 cm⁻¹˜1100 cm⁻¹ where an absorption peak is present, and contains a carboxyl group (—CO₂⁻) at a wave number of 1500 cm⁻¹˜1650 cm⁻¹ where an absorption peak is present. This is usually a characteristic of carbonates. The compound contains an amino group (NH₃⁺) at a wave number of 2800 cm⁻¹˜2000 cm⁻¹ where an absorption peak is present, and this is usually a characteristic of amine salts.

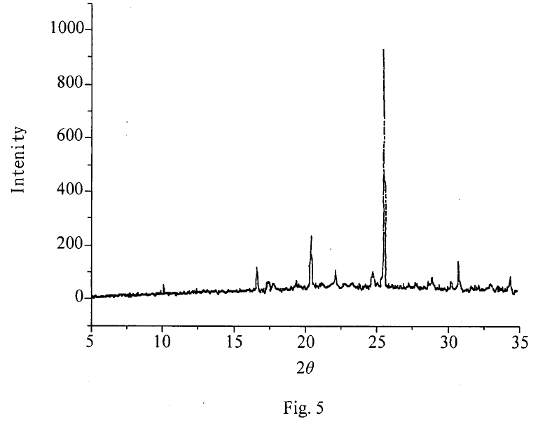

Referring to FIG. 5, there is depicted an X-ray powder diffractogram of an ibuprofen amine salt according to the present invention. X-ray powder diffraction is primarily used in qualitative and quantitative analysis of a crystalline phase and the determination of grain size and internal strain, residual stress analysis, the determination of structure, and crystallinity analysis, and so on. Basically, an X-ray diffraction of a crystal provides two items of important information: the first one is the diffraction peak position 2θ, and the second one is the diffraction peak intensity (I). The diffraction peak position gives the information on the morphology and size of a crystal cell and the lattice parameters. The diffraction peak intensity gives the information about the species and positions of the atoms within the crystal. With the variation of the crystal structure and composition of a material, these two items of information for each crystal are different. When material is analyzed by X-ray diffraction, different crystalline compounds produce combinations of different diffraction peak positions and diffraction peak intensities, which are referred to as diffraction patterns. From this drawing, it can be seen that the diffraction peak positions 20 of the ibuprofen amine salt are 21.5 degrees, 25 degrees, 26 degrees and 31 degrees, and the intensity is between 940 units and 960 units.

Additionally, X-ray single crystal diffraction analysis is used in order to realize the structure of ibuprofen amine salt according to the present invention, which is one of the most common methods for analyzing crystal structures. In an embodiment, after an ibuprofen amine salt under test is analyzed, the lengths of the sides of the cell obtained are a=17.578 angstroms (Å), b=10.428 angstroms (Å), and c=9.991 angstroms (Å) respectively. The relative angles of the cell sides are α=90.00° (degrees), β=97.174° (degrees), γ=90.00° (degrees), and space group: P2(1)/c. The cell has three diad axes perpendicular to one another so that it can be determined that the ibuprofen amine salt according to the present invention belongs to the monoclinic system, with approximately square morphology and the volume of the cell of 1817.0 Å³. These given conditions can be used to further obtain a density ranging from 1.1 mg/m³ to 1.3 mg/m³.

In an embodiment, in order to realize the solubility of the ibuprofen amine salt according to the present invention in water, about 30 mg to about 50 mg of the solid of ibuprofen amine salt was placed in a 20 ml measuring flask, and water was gradually dropped into the flask. Each time 50 microliters to 100 microliters of water had been dropped, the flask was shaken and the above operation was repeated until the solid in the flask just dissolved. The entire procedure was performed in water bath at 25° C. The solid weight of the ibuprofen amine salt and the volume of water used each time must be recorded in details. The solubility defined by dividing the total solid weight by the total volume (mg/ml) of water added to the flask can be used to calculate the solubility of the ibuprofen amine salt according to the present invention in water ranging from about 5.5 mg/ml to about 6.5 mg/ml.

As gathered from the foregoing, the ibuprofen amine salt according to the present invention has characteristics of the solubility in water ranging from 5.5 mg/ml to 6.5 mg/ml and the melting point ranging from 158° C. to 162° C., and has higher solubility in water and a higher melting point than common racemic mixtures of ibuprofen, and is stable between pH 4 and 9 after tested. Therefore, it has better bioavailability and properties than racemic mixtures of ibuprofen during pharmaceutical processing, and also has greater potential in long-acting drug applications.

The above-described embodiment is only illustrative but not limitative. Various equivalent modifications or changes to the present invention can be made to the elements of the present invention without departing from the spirit and scope of this invention. Accordingly, all such equivalent modifications and changes shall fall within the scope of the appended claims.

What is claimed is:

1. An ibuprofen amine salt, characterized in that said ibuprofen amine salt is yielded by the neutralization reaction of a racemic mixture of ibuprofen with tris(hydroxymethyl)amino-methane in a solution system of water and an organic solvent.

2. The ibuprofen amine salt as claimed in claim 1, wherein said ibuprofen amine salt is in a crystalline form and said crystal is white.

3. The ibuprofen amine salt as claimed in claim 2, wherein the aspect ratio of said crystal of said ibuprofen amine salt ranges from 0.9 to 1.1.

4. The ibuprofen amine salt as claimed in claim 1, wherein the solubility of said ibuprofen amine salt in water ranges from 5.5 mg/ml to 6.5 mg/ml.

5. The ibuprofen amine salt as claimed in claim 1, wherein the melting point of said ibuprofen amine salt ranges from 158° C. to 162° C.

6. The ibuprofen amine salt as claimed in claim 1, wherein the density of said ibuprofen amine salt ranges from 1.1 g/cm³ to 1.3 g/cm³.

7. The ibuprofen amine salt as claimed in claim 1, wherein the organic solvent comprises acetone, methanol, dimethyl sulfoxide, ethanol, N,N-dimethylformamide, acetonitrile, isopropyl alcohol, 1,4-dioxane or tetrahydrofuran.

8. A method of synthesizing an ibuprofen amine salt, comprising the following steps:
providing a racemic mixture of ibuprofen and an aqueous solution of tris(hydroxymethyl)-aminomethane;
dissolving a first number of moles of said racemic mixture of ibuprofen into an organic solvent to obtain a mixed solution;
gradually adding an aqueous solution of a second number of moles of tris(hydroxymethyl)-aminomethane to said mixed solution;
performing a stirring process and subsequently performing a separation process to obtain a precipitate;
performing a washing process on said precipitate with an aqueous solution containing an organic solvent; and
performing a drying process to obtain the ibuprofen amine salt.

9. The method of synthesizing an ibuprofen amine salt as claimed in claim 8, wherein said organic solvent comprises acetone, methanol, dimethyl sulfoxide, ethanol, N,N-dimethylformamide, acetonitrile, isopropyl alcohol, 1,4-dioxane or tetrahydrofuran.

10. The method of synthesizing an ibuprofen amine salt as claimed in claim 8, wherein said second number of moles is greater than said first number of moles.

11. The method of synthesizing an ibuprofen amine salt as claimed in claim 8, wherein said separation process includes filtration, centrifugation, or volatilization.

12. The method of synthesizing an ibuprofen amine salt as claimed in claim 8, wherein said precipitate is white.

13. The method of synthesizing an ibuprofen amine salt as claimed in claim 8, wherein said ibuprofen amine salt is in a crystalline form and the aspect ratio of said crystal ranges from 0.9 to 1.1.

14. The method of synthesizing an ibuprofen amine salt as claimed in claim 8, wherein the solubility of said ibuprofen amine salt in water ranges from 5.5 mg/ml to 6.5 mg/ml.

15. The method of synthesizing an ibuprofen amine salt as claimed in claim 8, wherein the melting point of said ibuprofen amine salt ranges from 158° C. to 162° C.

16. The method of synthesizing an ibuprofen amine salt as claimed in claim 8, wherein the density of said ibuprofen amine salt ranges from 1.1 g/cm³ to 1.3 g/cm³.

17. The method of synthesizing an ibuprofen amine salt as claimed in claim 8, wherein said aqueous solution containing an organic solvent comprises an aqueous solution of acetone, methanol, dimethyl sulfoxide, ethanol, N,N-dimethylformamide, acetonitrile, isopropyl alcohol, 1,4-dioxane or tetrahydrofuran.

18. A method of synthesizing an ibuprofen amine salt, comprising the following steps:
providing a racemic mixture of ibuprofen, tris(hydroxymethyl)aminomethane and an organic solvent;
adding a first number of moles of said racemic mixture of ibuprofen and a first number of moles of said tris(hydroxymethyl)-aminomethane to water to obtain a mixed solution, and placing said mixed solution in a constant temperature environment of a first temperature;
after adding a second number of moles of said organic solvent, performing a stirring process until the color of said mixed solution is near clear;
placing said mixed solution in a constant temperature environment of a second temperature until the crystal is crystallized from said mixed solution;
performing a separation process to obtain the crystal; and
performing a drying process to obtain the ibuprofen amine salt.

19. The method of synthesizing an ibuprofen amine salt as claimed in claim 18, wherein said organic solvent comprises acetone, methanol, dimethyl sulfoxide, ethanol, N,N-dimethylformamide, acetonitrile, isopropyl alcohol, 1,4-dioxane or tetrahydrofuran.

20. The method of synthesizing an ibuprofen amine salt as claimed in claim 18, wherein said second number of moles is greater than said first number of moles.

21. The method of synthesizing an ibuprofen amine salt as claimed in claim 18, wherein said separation process includes filtration, centrifugation, or volatilization.

22. The method of synthesizing an ibuprofen amine salt as claimed in claim 18, wherein said crystal is white.

23. The method of synthesizing an ibuprofen amine salt as claimed in claim 18, wherein said ibuprofen amine salt is in a crystalline form and the aspect ratio of said crystal ranges from 0.9 to 1.1.

24. The method of synthesizing an ibuprofen amine salt as claimed in claim 18, wherein the solubility of said ibuprofen amine salt in water ranges from 5.5 mg/ml to 6.5 mg/ml.

25. The method of synthesizing an ibuprofen amine salt as claimed in claim 18, wherein the melting point of said ibuprofen amine salt ranges from 158° C. to 162° C.

26. The method of synthesizing an ibuprofen amine salt as claimed in claim 18, wherein the density of said ibuprofen amine salt ranges from 1.1 g/cm³ to 1.3 g/cm³.
